(19) United States
(12) Patent Application Publication
(10) Pub. No.: US 2023/0184606 A1
(43) Pub. Date: Jun. 15, 2023

(54) SYSTEM FOR SUBSTRATE DETECTION AND MAPPING USING FORCE SENSING AND MAGNETIC COUPLING

(71) Applicant: Xerxes Maximilian Libsch, White Plains, NY (US)

(72) Inventor: Xerxes Maximilian Libsch, White Plains, NY (US)

(21) Appl. No.: 17/922,154

(22) PCT Filed: Apr. 28, 2021
(86) PCT No.: PCT/US2021/029748

§ 371 Date: Oct. 28, 2022

Related U.S. Application Data
(60) Provisional application No. 63/016,437, filed on Apr. 28, 2020.

(51) Int. Cl. G01L 5/00

(52) U.S. Cl. CPC G01L 5/0028

(57) ABSTRACT

The disclosed system relates to the device, system and methods for the characterization of a substrate based on force sensing. More specifically, force sensing is used to extract a force profile upon substrate insertion that allows for the characterization of the substrate along the axis of insertion of the force sensing probe. Force sensing can be provided by a load cell or strain gauge device or equivalent force sensing measure. The force system can contain a magnetic coupling method in order to provide contact between the probe and force sensing apparatus. The disclosed invention can be included in a system that includes hardware and software to process the data from the force sensor. The hardware and software can also be coupled with a data repository and corresponding methods in order to map real-time force sensing data with known force sensing data in order to provide positional information based on the particular known substrate.

### CROSS-REFERENCE TO RELATED APPLICATIONS

The present application is the nationalization of PCT Application PCT/US2021/029748, filed Apr. 28, 2021, published as WO2021222471A1 on Nov. 11, 2021, said application incorporated herein by reference in its entirety. PCT/US2021/029748 is an international PCT application of and claims the benefit of U.S. Provisional Application No. 63/016,437, filed Apr. 28, 2020, which is incorporated herein by reference in its entirety.

### BACKGROUND OF THE INVENTION

There are several prior art devices.

Cochlear Electrode Insertion US8594799B2. Described is a system and method for mechanically assisted insertion of an electrode that includes an insertion tool and utilizes force profile data in conjunction with force sensing to determine positional information and gauge insertion success.

Insertion Mode Phacoemulsification Employing Powered Iol Delivery, WO2011133853A1. Described is a system and method for an ocular surgical procedure using an intraocular lens to an eye through an incision in the eye. In addition, the system and method includes monitoring of incisions based on force encountered and uses feedback of the force encountered in order to selectively alter the force applied.

Force Measurement Apparatus and Force Measurement Method, Master Slave Apparatus, Force Measurement Program and Integrated Electronic Circuit, US20150057575A1. Described is a system and method for force measurement apparatus that measures force during insertion time into a living body vessel.

Implant Insertion Device, US20070149981A1. Described is a system and method for controlling the amount of force applied to an implant or other element. The system includes an inserter tool that has a force controlling element coupled to the shaft. The system is capable of measuring the amount of force applied to the inserter tool.

Method For Manufacturing a Needle That Includes an Aligned Magnetic Element, ES2739882T3. Described is a system and method for joining a magnetic element to a needle assembly, which includes a cannula.

Use of Position and Force Measurements To Estimate Tissue Thickness, JP6320721B2. Described is a system and method for using force to detect the thickness of tissue in the body.

System and Method for Evaluating Tissue, US8845555B2. Described is a system and method for characterizing tissue to check for abnormal growths by applying force to a particular area of the tissue and measuring corresponding displacements in other areas of the tissue.

Nonlinear System Identification Techniques and Devices for Discovering Dynamic and Static Tissue Properties, US8758271B2. Described is a system and method for measuring mechanical properties of a tissue that includes a probe configured to perturb the tissue, and a plurality of force sensors to measure the response of the perturbation.

Multi-Force Sensing Surgical Instrument and Method of Use for Robotic Surgical Systems, US9549781B2. Described is a system and method for a force sensing instrument that includes a tool with a shaft that has a plurality of strain gauge and force sensors in order to determine the magnitude and position of a lateral component of force on the tool.

Method for Presenting Force Sensor Information Using Cooperative Robot Control and Audio Feedback, US20140052150A1. Described is a system and method for cooperative control of surgical tool that includes a holder as well as force sensor for sensing force due to operator input and tool tip forces.

In general, a wide array of applications benefit from being able to characterize a particular substrate, especially applications that require mechanical puncture or insertion into the particular substrate.

In the field of robotics, such applications are particularly ubiquitous. A common problem is using sensors to characterize a particular substrate. In particular, if mechanical puncture into the substrate is needed, a method for positional detection within the substrate is required. Such is the case for example if a robot must insert a probe into a particular region of the substrate. This occurs for example in agriculture devices that require soil probes to be taken. Different soils have different properties that affect the insertion force required to insert the probe into the soil. Characterization of this force is thus an important factor in determining the soil properties and mapping the soil properties to those of known quantities is beneficial in characterizing the environment.

In the field of medical devices and procedures, such applications are characteristically common as well. Surgical procedures involve insertion of a mechanical probe into a particular substrate and require knowledge of positional information within that substrate. Such could be the case for a procedure that requires inserting a needle (probe) into a vein. The procedure requires positional information so that the precise stopping point required can be achieved. This for example, can be the stopping of a needle within a vein. The substrate has different properties and layers which can be characterized by the force needed for probe insertion into the different layers. Thus, characterization of the force is an important factor in determining substrate properties and can be compared with known substrate properties in order to obtain positional information of the probe within the substrate.

In addition, a common problem that is present with force-based feedback systems is the ability to couple a probe to the system without inducing bias. Rigid attachment, such as those provided by fixed coupling systems inherently introduce a force bias into the system which can skew the performance of the force feedback system. Thus, it is of great need to provide a coupling system for attachment probes to a device that uses force measurements and does not induce bias, but still allows for precise alignment.

In U.S. Pat. No. US8845555B2, the invention provides a sensor system for characterizing tissue to check for abnormal growths by applying force to a particular area of the tissue and measuring corresponding displacements in other areas of the tissue. However, the device does not concern probe insertion into the tissue and does not utilize force feedback to measure force measurements from the probe.

In U.S. Pat. No. US8594799B2, a system and method for mechanically assisted insertion of an electrode that includes a tool to utilize force profile data is described. However, the system does not provide magnetic coupling methods to address the problem of mechanical induced force bias and only utilizes maximum allowable force profiles during electrode insertion.

In U.S. Pat. No. US9549781B2, a force sensing instrument that includes a tool with a shaft that has a plurality of strain gauge and force sensors in order to determine the magnitude and position of a lateral component of the force on the tool is described. However, the invention does not concern force normal to the insertion substrate and does not solve the problem of mechanical induced force bias.

Despite prior approaches, there still exist several limitations that exist in the prior art. First, there exists a greet need for a device system and methodology with a greater range of performance and generality that allows for substrate characterization based on force sensing during insertion into the substrate and that employs a method of non-fixed coupling to couple the force sensing system and insertion probe in order to obtain coupling between the force sensing system and the insertion probe. The object of this invention is to address these limitations in the prior art.

### SUMMARY OF THE INVENTION

The disclosed system relates to the device, system and methods for the characterization of a substrate based on force sensing. More specifically, force sensing is used to extract a force profile upon substrate insertion that allows for the characterization of the substrate along the axis of insertion of the force sensing probe. Force sensing can be provided by a load cell or strain gauge device or equivalent force sensing measure. The force system can contain a magnetic coupling method in order to provide contact between the probe and force sensing apparatus. The disclosed invention can be included in a system that includes hardware and software to process the data from the force sensor. The hardware and software can also be coupled with a data repository and corresponding methods in order to map real-time force sensing data with known force sensing data in order to provide positional information based on the particular known substrate.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1: is a schematic of the device according to an embodiment of the present invention, for substrate detection and mapping using force sensing and magnetic coupling of a probe.

FIG. 2: is a schematic of the system according to an embodiment of the present invention, for substrate detection and mapping using force sensing and magnetic coupling of a probe.

FIG. 3a: is a schematic of the methodology according to an embodiment of the present invention, for substrate detection and mapping using force sensing and magnetic coupling of a probe.

FIG. 3b: is a schematic of the methodology according to an embodiment of the present invention, for substrate detection and mapping using force sensing and magnetic coupling of a probe.

FIG. 3c: is a schematic of the methodology according to an embodiment of the present invention, for substrate detection and mapping using force sensing and magnetic coupling of a probe.

FIG. 4: is a diagram showing the representative force output based on substrate properties as a probe is inserted into the substrate.

### DETAILED DESCRIPTION OF THE INVENTION

### Device - Embodiment 1

The ultimate goal of this invention is to address the fundamental limitations aforementioned. One or more embodiments of the invention address these and other needs by providing a fundamentally different approach to substrate mapping using force sensing technology and a non-rigidly attached magnetic coupling system. This is shown in EMBODIMENT 1, which contains a base modular mounting structure that is hexagonal in shape and has a long mounting shaft cavity in order to be mounted onto another device, such as for positioning of the modular mounting structure. The base modular mounting structure contains a force probe, which is rigidly coupled to it by means of a screw attachment. Coupled to this force sensor, away from the base mounting structure, is a probe that contains a hollow channel allowing for the flow of liquid through the channel. The probe is magnetically coupled to the structure and force sensor such that accurate and repeatable alignment is achieved, but avoids fixed mechanical fixturing such causes sensor offset and sensor error, and would otherwise require the system to be calibrated to obtain zero offset error. This is achieved by means of a magnetic collet holder coupling, which couples with a magnetic collet, which in turn is coupled with the hollow probe.

### System - Embodiment 2

One embodiment of the present invention is shown in EMBODIMENT 2. The device of EMBODIMENT 1 is augmented. The base modular mounting structure is coupled to linear and rotational motion by means of two motors and a lead screw rail and carriage system to convert one of the motors’ rotary motion into linear motion. In addition, the base modular mounting structure contains a force sensor that is coupled to the mounting structure, as well as a probe that contains a hollow channel allowing for the flow of liquid through the channel. The probe is magnetically coupled to the structure and force sensor such that accurate and repeatable alignment between the two is achieved. In addition, the system contains a microcontroller that can contain data as well as algorithms. The microcontroller is housed in a base unit that additionally provides structural stability.

### Process - Embodiment 3a

Another embodiment of the present invention relates to the process by which encoding sensors may be used in order to determine the position or trajectory of each axis relating to rotolinear motion. At each time or motion step for the system in EMBODIMENT 2, sensor output from the encoding sensor along each output is used. A difference between the current step and previous step for the roto-linear device from EMBODIMENT 1 is used in order to obtain the current position of each axis. This is found by either absolute positional information received by the encoders, or by incrementally recording positional changes as each motion axis is varied.

### Process - Embodiment 3b

In addition, the embodiment of the present invention relates a methodology that may utilize an algorithm, and a force sensor to position the probe to an arbitrary position using roto linear motion to the surface of a substrate. For each encoding sensor, local positioning of each axis is obtained by comparing encoding sensor differentials at each point along the motion path. In addition, force sensor output is recorded, such that upon contact of the probe from EMBODIMENT 1 with the substrate, the probe transfers a force to the force sensor sufficiently attached to the modular platform. Using this force sensor data in addition to the encoding sensors thus allows for the position of the substrate to be found with respect to the axes of roto-linear motion.

### Process - Embodiment 3c

In addition, the embodiment of the present invention relates to a methodology that may utilize an algorithm as well as pre-tabulated force sensor data in order to determine position of the probe within a substrate using the force sensor. Using pre-tabulated data of known force outputs upon insertion of a probe into a substrate, and comparing the known signal with the force sensor output allows for a mapping between force signal and position within the substrate to be utilized in order to determine the position of the probe within a particular substrate. Thus, this allows for the global positioning of the probe within the substrate to be known. Finally, the encoding sensor signals as well as force sensing signal can be utilized to find the positional distance along a certain set of axes to a target position within the substrate, utilizing known mapping data between force profile signal and position within the substrate to find the error to an arbitrary target within the substrate.

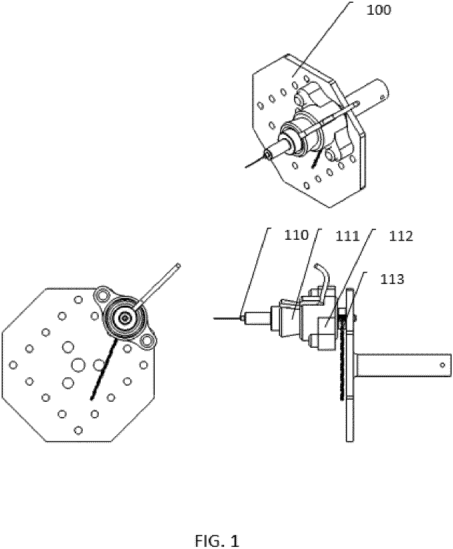

A further description of the example embodiments of the invention follows. Embodiments of the claimed invention can be first explained with reference to FIG. 1.

FIG. 1 is a schematic of the device according to an embodiment of the present invention. The device contains a base modular mounting structure (103) with dimensions 1 mm2 to 10,000 m2 that is functionally connected to at least one or more other hardware units. This includes a force sensor (100), which can be capable of measuring in the range 1 mN to 10,000 N. This force sensor is coupled to the modular mounting structure and contains a magnetically coupled probe. The force sensor is mounted directly to the base modular mounting structure by means a threaded screw system. The force sensor contains a thread screw system on the positive end (outward facing), to which a magnetic fixture is tapped and attached (112). The magnetic fixture, named the collet holder, allows for subsequent contact with a magnetic collet insert (111) attached to a probe (110) that contains a channel for the flow of liquids to flow through to be attached non-permanently. This allows for repeatable and accurate alignment that does not induce bias to force sensing signals that would have otherwise resulted from rigid attachment.

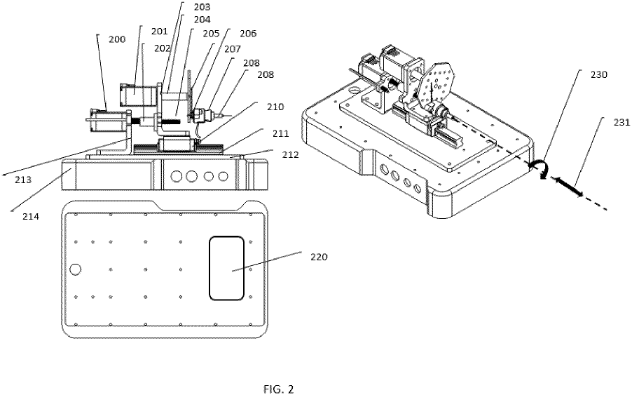

A further description of the invention can be explained with reference to FIG. 2.

In FIG. 2, the device from FIG. 1 is augmented to include a positioning system, such that the modular mounting structure (204) can be positioned along two axes, one linear (231) and one rotary (230). The modular mounting platform is in turn attached to the output shaft of a motor (201) which allows it to turn in a rotary fashion along a central axis (230). This motor is then coupled to a mechanical fixture (203) which holds it rigidly to a linear carriage (210) by means of screw-based attachment. The linear carriage is free to slide along a linear rail (211), which is itself mounted to a baseplate (212) which supports the entire device. The linear rail is driven by a lead screw and nut system (202), which converts rotary motion from the output shaft of another motor (200) into linear motion along a given linear axis (231). This motor is in turn attached to the baseplate by means of a mechanical fixture (213). Everything, including the motors (200, 201), screw and nut drive (202), mechanical fixtures to hold the motors (203, 213), linear rail and carriage (210, 211), modular mounting structure (205), force sensor (206), magnetic fixture (207) and magnetic collet around a hollow probe (207, 208) is attached to a base mounting structure (214). This base mounting structure houses a microcontroller (220).

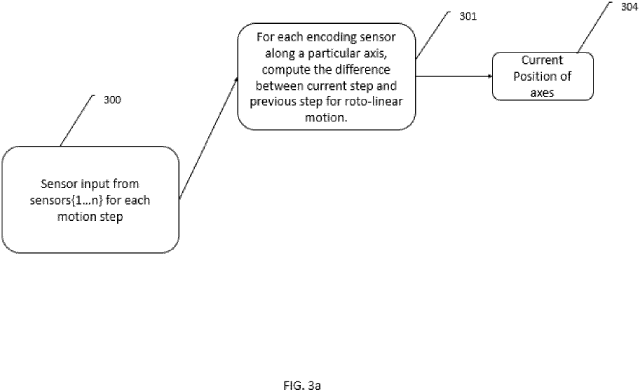

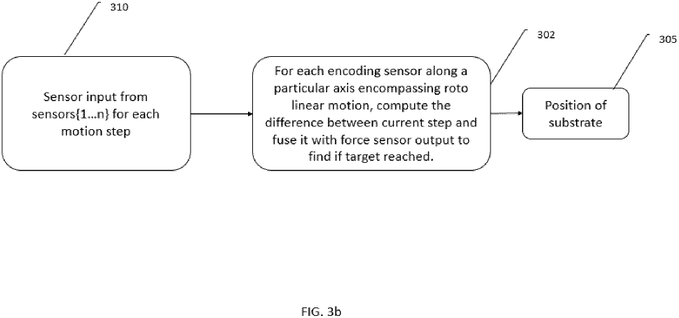

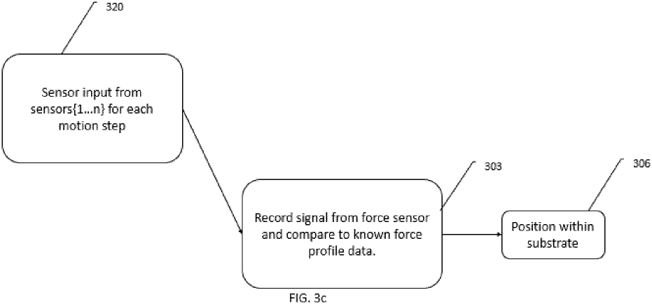

A further description of the invention can be explained with reference to FIG. 3a.

FIG. 3a is a schematic of the system according to an embodiment of the present invention showing the methodology by which the current position of each axis of motion relating to EMBODIMENT 1 (304). Sensor input from each sensor, including force sensors and encoding sensors is collected for each motion step or time step (300). For each encoding sensor along a particular axis, the difference between current step and previous step is computed in order to generate the current position of each axis (301).

A further description of the invention can be explained with reference to FIG. 3b.

FIG. 3b is a schematic of the system according to an embodiment of the present invention showing the methodology by which position of the substrate with respect to each axis in EMBODIMENT 1 can be found (305). Sensor input from each sensor, including force sensors and encoding sensors is collected for each motion step or time step (310). In addition, for each encoding sensor along a particular axis, the encoding sensor data is compared with force sensor data, such that when the probe sufficiently attached to the force sensor data and modular platform touches a particular substrate, the force sensor data registers this change in force resulting from the contact with the substrate, and the position of the substrate is thus found (302). This is found by relating a known distance from the probe tip to the base modular platform, which is thus the distance from the substrate surface to the base modular platform.

A further description of the invention can be explained with reference to FIG. 3c.

FIG. 3c is a schematic of the system according to an embodiment of the present invention showing the methodology by which the position of the probe with respect to EMBODIMENT 1 can be found (306). Sensor input from each sensor, including force sensors and encoding sensors is collected for each motion step or time step (320). In addition, the force sensor signal is recorded while the probe is inserted into the substrate, such that the force data can be compared with known force sensor data and thus a position within the substrate can be found through the force related to position mapping. Note that those skilled in the art would recognize that one or more methodologies encompassing, but not limited to those described in FIG. 3a, FIG. 3b, and FIG. 3c can be used simultaneously or independently. Such methodologies can also be implemented with feedback to each other, and that other feedback methodologies may be incorporated.

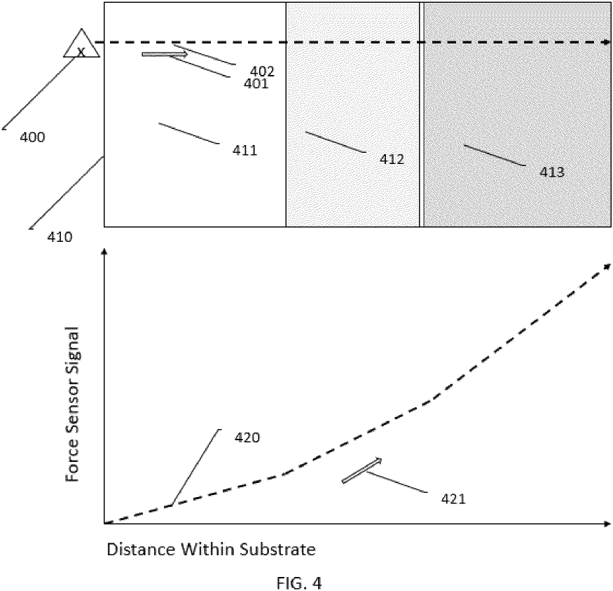

A further description of the invention can be explained with reference to FIG. 4.

FIG. 4 shows the representative output from the force sensor coupled to the modular mounting structure and coupled to the probe non-permanently from EMBODIMENT 1. The probe tip (400) is inserted into the substrate (410), which has three distinct areas (411, 412, 413). As the probe moves through the substrate (402) in a linear direction (401), the force output from the force sensor changes distinctly (420), which allows for methodology for obtaining a mapping between force and position.

While this invention has been particularly shown and described with reference to example embodiments thereof, it will be understood by those skilled in the art that various changes in form and details may be made therein without departing from the scope of the invention encompassed by the appended claims.

All references cited herein are incorporated herein by reference to the full extent allowed by law. The discussion of those references is intended merely to summarize the assertions made by their authors. No admission is made that any reference (or a portion of any reference) is relevant prior art. Applicants reserve the right to challenge the accuracy and pertinence of any cited reference.

What is claimed is:

1. A measuring device comprising:
a base modular mounting structure having a dimension from about 1 mm² to 10,000 m², which allows the the measuring device to be functionally connected to one or more other hardware units;
at least one or more force sensors, load cells or similar strain gauge sensor that is coupled to the modular mounting structure; and
a probe or similar object that is coupled to the force sensor non-permanently.

2. The device according to claim 1, wherein the force sensor is capable of measuring in the range 1 mN to 10,000 N.

3. The device according to claim 1, wherein the base modular mounting structure is attached to at least one or more movable axes of motion.

4. The device according to claim 1, wherein the probe is coupled to the force sensor using magnetic based attachment methods.

5. The device according to claim 1, wherein the probe is inserted into a substrate from 1 degree to 179 degrees with respect to the local tangent plane of the substrate.

6. The device according to claim 1, wherein the probe contains a channel for the flow of liquids to flow in either direction along the channel.

7. A measurement system comprising:
a base modular mounting structure having a dimension from about 1 mm² to 10,000 m², which allows the measuring device to be functionally connected to one or more other hardware units;
at least one or more force sensors, load cells or similar strain gauge sensor that is coupled to the modular mounting structure;
a probe or similar object that is coupled to the force sensor non-permanently;
one or more axes of motion that functionally transfer motion to the base modular mounting structure; and
allowances for containing feedback between the force sensor and each axis of motion.

8. The system according to claim 7, wherein the force sensor is capable of measuring in the range 1 mN to 10,000 N.

9. The device according to claim 1, wherein the axes of motion can be rotational or linear in nature.

10. The system according to claim 7, wherein the probe is coupled to the force sensor using magnetic based attachment methods.

11. The system according to claim 7, wherein the probe is inserted into a substrate from 1 degree to 179 degrees with respect to the local tangent plane of the substrate.

12. The system according to claim 7, wherein the base modular mounting structure might be functionally attached to one or more axes of motion.

13. The system according to claim 7, wherein the system includes a microcontroller or similar means of handling input, output and data processing.

14. The system according to claim 7, wherein the microcontroller might incorporate memory, software or algorithms.

15. The system according to claim 7, wherein the feedback can be controlled using software and algorithms.

16. A method of measuring comprising:
a base modular mounting structure having a dimension from about 1 mm² to 10,000 m², which allows the measuring device to be functionally connected to one or more other hardware units;
at least one or more force sensors, load cells or similar strain gauge sensor that is coupled to the modular mounting structure; and
a probe or similar object that is coupled to the force sensor non-permanently.
a methodology that may utilize the encoding sensors to determine positional information of the probe.

17. The methodology according to claim 16 wherein the methodology is iterative based on a time frame between 1 nanosecond and 1 hour.

18. The methodology according to claim 16, wherein the base modular mounting structure is attached to at least one or more movable axes of motion.

19. The methodology according to claim 16, wherein encoding sensors corresponding to motion axes are used in a feedback loop.

20. A method of measuring comprising:
a base modular mounting structure having a dimension from about 1 mm² to 10,000 m², which allows the the measuring device to be functionally connected to one or more other hardware units;
at least one or more force sensors, load cells or similar strain gauge sensor that is coupled to the modular mounting structure; and
a probe or similar object that is coupled to the force sensor non-permanently.
a methodology that at least uses data or memory in order to determine positional information of the substrate.

21. The methodology according to claim 20 wherein the methodology is iterative based on a time frame between 1 nanosecond and 1 hour.

22. The methodology according to claim 20, wherein the base modular mounting structure is attached to at least one or more movable axes of motion.

23. The methodology according to claim 20, wherein encoding sensors corresponding to motion axes are used.

24. The methodology according to claim 20, wherein the force sensor is used in a feedback loop.

25. A method of measuring comprising :
a base modular mounting structure having a dimension from about 1 mm² to 10,000 m², which allows the the measuring device to be functionally connected to one or more other hardware units;
at least one or more force sensors, load cells or similar strain gauge sensor that is coupled to the modular mounting structure;
a probe or similar object that is coupled to the force sensor non-permanently; and
a methodology that at least uses data or memory in order to determine distance of the probe to a desired target within a given substrate.

26. The methodology according to claim 25 wherein the methodology is iterative based on a time frame between 1 nanosecond and 1 hour.

27. The device according to claim 1, wherein the base modular mounting structure is attached to at least one or more movable axes of motion.

28. The methodology according to claim 25, wherein encoding sensors corresponding to motion axes are used.

29. The methodology according to claim 25, wherein the force sensor is used in a feedback loop.
